(19) United States
(12) Patent Application Publication
(10) Pub. No.: US 2021/0076964 A1
(43) Pub. Date: Mar. 18, 2021

(54) IDENTIFYING P WAVE OVERSENSING

(71) Applicant: Cardiac Pacemakers, Inc., St. Paul, MN (US)

(72) Inventors: Deepa Mahajan, North Oaks, MN (US); David L. Perschbacher, Coon Rapids, MN (US); Derek D. Bohn, Woodbury, MN (US)

(21) Appl. No.: 17/022,375

(22) Filed: Sep. 16, 2020

Related U.S. Application Data
(60) Provisional application No. 62/901,023, filed on Sep. 16, 2019.

(51) Int. Cl. A61B 5/0456; A61B 5/0452; A61N 1/37

(52) U.S. Cl. CPC A61B 5/0456; A61N 1/3704; A61N 1/3706; A61B 5/04525

(57) ABSTRACT

Systems and methods to determine P wave oversensing (PWOS) are disclosed, including identifying cardiac signal features in received cardiac electrical information, determining a first indication of PWOS using a pattern of identified cardiac signal features, and in response to the determined first indication of PWOS, determining a second indication of PWOS using a morphology of the received cardiac electrical information.

### CLAIM OF PRIORITY

This application claims the benefit of priority under 35 U.S.C. § 119(e) of U.S. Provisional Patent Application Ser. No. 62/901,023, filed on Sep. 16, 2019, which is herein incorporated by reference in its entirety.

### TECHNICAL FIELD

This document relates generally to medical devices, and more particularly, but not by way of limitation, to systems, devices, and methods for identifying P wave oversensing (PWOS) in medical devices.

### BACKGROUND

Ambulatory medical devices (AMDs), including implantable, subcutaneous, wearable, or one or more other medical devices, etc., have been used to monitor, detect, or treat various conditions, including heart failure (HF), atrial fibrillation (AF), etc. AMDs may include sensors to sense physiological signals from a patient. Frequent patient monitoring and early detection of worsening patient condition, including worsening heart failure (WHF) or AF, may help improve patient outcome. Identification of patients or groups of patients at an elevated risk of future adverse events may help provide timely patient treatment or prevent or reduce patient hospitalization. Identifying and safely managing patient risk of worsening condition may avoid unnecessary medical interventions or hospitalizations and reduce healthcare costs.

AMDs can be configured to receive physiologic information, including cardiac electrical information, associated with various implantable or external locations of a patient. For example, certain AMDs are configured to receive cardiac electrical information from implantable electrodes located within or on the heart, including coupled to a lead and located in one or more chambers of the heart or within the vasculature of the heart near one or more chambers. Certain AMDs include one or more atrial leads, or receive information from one or more atrial leads, configured to be located in a right atrium of a patient. Other AMDs do not include an atrial lead or electrode in the right atrium. Detection of atrial events, such as atrial depolarizations, can be challenging without information from an atrial lead or an electrode in the right atrium.

### SUMMARY

This document discusses, among other things, systems and methods to determine P wave oversensing (PWOS), including identifying cardiac signal features in received cardiac electrical information, determining a first indication of PWOS using a pattern of identified cardiac signal features, and in response to the determined first indication of PWOS, determining a second indication of PWOS using a morphology of the received cardiac electrical information.

In Example 1, subject matter (e.g., a system) may comprise: a signal receiver circuit configured to receive cardiac electrical information of a patient; an assessment circuit configured to: identify cardiac signal features in a first portion of the received cardiac electrical information; determine a first indication of P wave oversensing (PWOS) in the first portion of the received cardiac electrical information using a pattern of identified cardiac signal features; and in response to the determined first indication of PWOS in the first portion of the received cardiac electrical information, determine a second indication of PWOS in the first portion of the received cardiac electrical information using a morphology of the first portion of the cardiac electrical information.

In Example 2, the subject matter of Example 1 may optionally be configured such that the first portion of the received cardiac electrical information includes a first cardiac cycle of the patient, and the cardiac signal features include at least one of a P wave or an R wave of the first cardiac cycle.

In Example 3, the subject matter of any one or more of Examples 1-2 may optionally be configured such that the first portion of the received cardiac electrical information includes multiple cardiac cycles of the patient, and the cardiac signal features include at least one of a P wave of the multiple cardiac cycles or an R wave of the multiple cardiac cycles.

In Example 4, the subject matter of any one or more of Example 1-3 may optionally be configured such that the pattern of identified cardiac signal features includes a pattern of amplitudes of the identified cardiac signal features or timings of or between the identified cardiac signal features.

In Example 5, the subject matter of any one or more of Example 1-4 may optionally be configured such that the pattern of identified cardiac signal features includes a pattern of timings between identified cardiac signal features.

In Example 6, the subject matter of any one or more of Example 1-5 may optionally be configured such that, to determine the second indication of PWOS, the assessment circuit is configured to compare a morphology of at least one identified cardiac signal feature associated with the determined indication of PWOS to a template for the identified cardiac signal feature to confirm the determined first indication of PWOS.

In Example 7, the subject matter of any one or more of Example 1-6 may optionally be configured such that the template includes at least one of an R wave template or a P wave template.

In Example 8, the subject matter of any one or more of Example 1-7 may optionally be configured such that the system is a medical-device system, comprising: a cardiac stimulation circuit configured to provide a cardiac stimulation signal to stimulate a heart of the patient; and a stimulation control circuit configured to adjust the cardiac stimulation signal using the determined second indication of PWOS in the first portion of the received cardiac electrical information.

In Example 9, the subject matter of any one or more of Example 1-8 may optionally be configured to comprise: multiple electrodes configured to provide stimulation to the heart of the patient using the cardiac stimulation signal from the cardiac stimulation circuit and to detect the cardiac electrical information of the patient.

In Example 10, subject matter (e.g., a method) may comprise: receiving, using a signal receiver circuit, cardiac electrical information of a patient; identifying, using an assessment circuit, cardiac signal features in a first portion of the received cardiac electrical information; determining, using the assessment circuit; a first indication of P wave oversensing (PWOS) in the first portion of the received cardiac electrical information using a pattern of identified cardiac signal features; and in response to the determined first indication of PWOS in the first portion of the received cardiac electrical information, a second indication of PWOS in the first portion of the received cardiac electrical information using a morphology of the first portion of the cardiac electrical information.

In Example 11, the subject matter of Example 10 may optionally be configured such that the first portion of the received cardiac electrical information includes a first cardiac cycle of the patient, and the cardiac signal features includes at least one of a P wave or an R wave of the first cardiac cycle.

In Example 12, the subject matter of any one or more of Example 10-11 may optionally be configured such that the first portion of the received cardiac electrical information includes multiple cardiac cycles of the patient, and the cardiac signal features include at least one of a. P wave of the multiple cardiac cycles or an R wave of the multiple cardiac cycles.

In Example 13, the subject matter of any one or more of Example 10-12 may optionally be configured such that the pattern of identified cardiac signal features includes a pattern of amplitudes of the identified cardiac signal features or timings of or between the identified cardiac signal features.

In Example 14, the subject matter of any one or more of Example 10-13 may optionally be configured such that the pattern of identified cardiac signal features includes a pattern of timings between identified cardiac signal features.

In Example 15, the subject matter of any one or more of Example 10-14 may optionally be configured such that determining the second indication of PWOS includes comparing a morphology of at least one identified cardiac signal feature associated with the determined indication of PWOS to a template for the identified cardiac signal feature to confirm the determined first indication of PWOS.

In Example 16, the subject matter of any one or more of Example 10-15 may optionally be configured such that the template includes at least one of an R wave template or a P wave template.

In Example 17, the subject matter of any one or more of Example 10-16 may optionally be configured to comprise: providing, using a cardiac stimulation circuit, a cardiac stimulation signal to stimulate a heart of the patient; and adjusting, using a stimulation control circuit, the cardiac stimulation signal using the determined second indication of MOS in the first portion of the received cardiac electrical information.

In Example 18, the subject matter of any one or more of Example 10-17 may optionally be configured to comprise: providing, using multiple electrodes, stimulation to the heart of the patient using the cardiac stimulation signal from the cardiac stimulation circuit; and detecting the cardiac electrical information of the patient using the multiple electrodes.

In Example 19, subject matter (e.g., a system) may comprise: a signal receiver circuit configured to receive cardiac electrical information of a patient; an assessment circuit configured to: identify cardiac signal features in a first portion of the received cardiac electrical information, wherein the first portion includes a first cardiac cycle of the patient and the cardiac signal features include at least one of a P wave or an R wave of the first cardiac cycle; determine a first indication of P wave oversensing (PWOS) in the first portion of the received cardiac electrical information using a pattern of identified cardiac signal features; and in response to the determined first indication of PWOS in the first portion of the received cardiac electrical information, determine a second indication of PWOS in the first portion of the received cardiac electrical information using a morphology of the first portion of the cardiac electrical information.

In Example 20, the subject matter of Example 19 may optionally be configured such that: the pattern of identified cardiac signal features includes a pattern of amplitudes of the identified cardiac signal features or timings of or between the identified cardiac signal features; to determine the second indication of PWOS, the assessment circuit is configured to compare a morphology of at least one identified cardiac signal feature associated with the determined indication of PWOS to a template for the identified cardiac signal feature to confirm the determined first indication of MOS; and the template includes at least one of an R wave template or a P wave template.

In Example 21, subject matter (e.g., a system or apparatus) may optionally combine any portion or combination of any portion of any one or more of Examples 1-20 to comprise “means for” performing any portion of any one or more of the functions or methods of Examples 1-20, or at least one “non-transitory machine-readable medium” including instructions that, when performed by a machine, cause the machine to perform any portion of any one or more of the functions or methods of Examples 1-20.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates example relationships between features of a cardiac electrical signal of a patient.

FIG. 2 illustrates an example system including a signal receiver circuit and an assessment circuit.

FIG. 3 illustrates an example patient management system and portions of an environment in which the system may operate.

FIG. 4 illustrates an example method of detecting P wave oversensing (PWOS).

FIG. 5 illustrates an example relationship between features of a cardiac signal of a patient.

FIG. 6 illustrates a block diagram of an example machine upon which any one or more of the techniques discussed herein may perform.

### DETAILED DESCRIPTION

Certain medical events or potential medical events, such as arrhythmia or potential arrhythmia events (e.g., AF, etc.), can be detected using cardiac electrical information, including, for example, detected atrial or ventricular events (e.g., beats, r-waves, p-waves, etc.), intervals therebetween, or intervals between the detected atrial or ventricular events and one or more other physiological events (e.g. one or more heart sound events, etc.). Cardiac electrical information, including a cardiac electrical signal, can be detected using one or more sensors, including, for example, one or more electrodes, such as associated with one or more leads, etc.

AMDs can be configured to receive cardiac electrical information of a patient, detect cardiac events using the received cardiac electrical information, and determine a patient condition, including the presence or absence of an arrhythmia event or potential arrhythmia event, using the detected cardiac events. The cardiac electrical signal can include one or more features, such as the P wave, a QRS complex, a T wave, etc., each representative of different physiologic information. For example, the P wave can represent depolarization of the atria, or atrial systole. The QRS complex can represent depolarization of the ventricles, or ventricular systole, and can include three parts: a Q wave, an R wave, and an S wave. The Q wave can represent depolarization of the interventricular septum. The R wave can represent the main electrical stimulus as it passes through the main portion of the ventricles. The S wave can represent depolarization of the Purkinje fibers. The T wave can represent repolarization of the ventricles.

The present inventors have recognized, among other things, improved systems and methods for identifying and mitigating P wave oversensing (MOS), or misidentification of the P wave as one or more other cardiac electrical signal features or parameters, such as an R wave, etc., in AMDs or medical device systems configured to sense or detect a P wave or an R wave in a cardiac electrical signal or cardiac electrical information. In an example, (1) a first, initial determination of PWOS using patterns of detected features of a cardiac electrical signal, such as detected abnormal timings or amplitudes of or between detected P waves or R waves, etc., and (2) a separate, second confirmation of such first initial determination of PWOS, such as a morphology analysis of the first initial determination. In certain examples, the first initial determination can require less resources (e.g., power, time, etc.) than the separate, second confirmation, while providing the sensitivity and specificity of the separate, second confirmation. Further, the separate, second confirmation can provide more sensitivity and specificity than the first initial determination. Accordingly, the specific, staged combination (e.g., the first, then the second only if the first) may provide distinct advantages over each separately or both together.

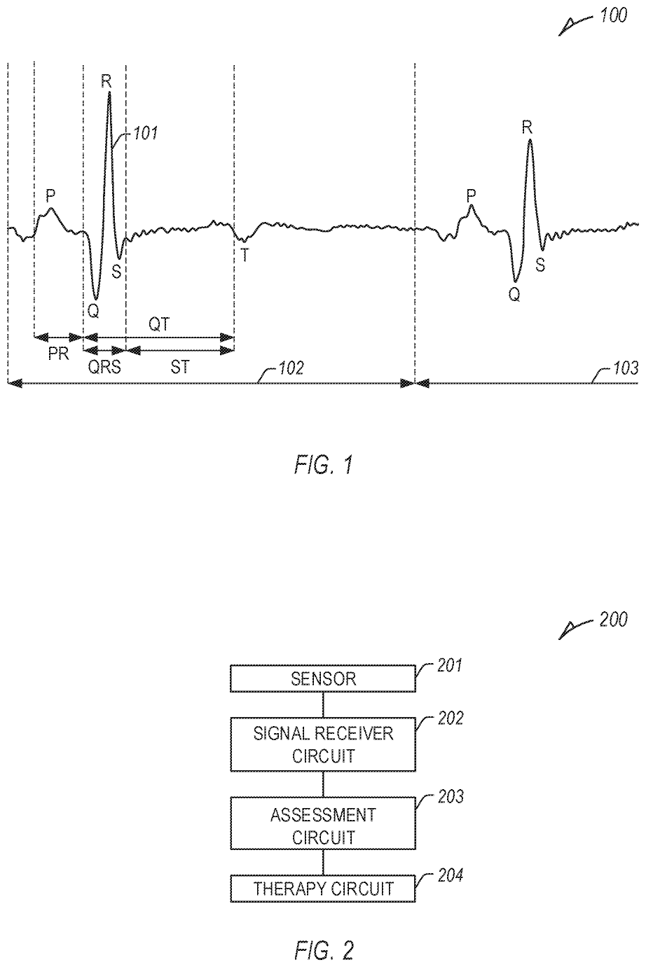

FIG. 1 illustrates example relationships 100 between features of a cardiac electrical signal 101 of a patient over multiple physiologic cycles of the patient including first and second cardiac cycles 102, 103. The cardiac electrical signal 101 can include an electrogram (EGM) or an electrocardiogram (ECG) signal. EGM generally refers to an intracardiac electrogram, a cardiac electrical signal sensed internally within the patient, whereas ECG generally refers to a cardiac electrical signal sensed externally, on the skin or at the surface of a patient. The cardiac electrical signal 101 includes different cardiac electrical features or parameters, including the P wave, the QRS complex (including the Q wave, the R wave, and the S wave), the T wave, etc. The relationships 100 can further include different timing intervals between different features of the cardiac electrical signal 101, including a PR interval, a QRS interval, a QT interval, an ST interval, etc. In other examples, features of the cardiac electrical signal 101 can be measured in relation to other electrical or mechanical physiologic signals, such as respiration or heart sounds, etc.

PWOS can be problematic in medical device systems and methods, as blanking periods, refractory periods, or detection thresholds are often employed in relation to detection of various electrical features or parameters of the cardiac electrical signal 101. For example, if a blanking interval is triggered from the P wave instead of after the QRS complex, an ensuing T wave can be mis-identified. If a P wave is improperly identified as an R wave, the ensuring R wave may be missed or detected as an additional R wave. Such misidentification can add error or noise to individual, averaged, trended, or combined values or intervals, which may affect other detections or determinations, including detection or trending of a PR interval (an interval between the P wave and the R wave of one physiologic cycle) or one or more other measures or intervals, or determination of one or more indications of patient conditions, such as AF, etc.

A continuous morphology analysis can be used to distinguish P waves from R waves (and vice versa), but such morphology analysis is problematic, as continuous morphology analysis can be resource intensive (e.g., power, time, and computationally), especially problematic in AMDs with limited power supply, or devices where recharging or replacing power supplies (e.g., implantable medical devices) is burdensome. Accordingly, there is a need for more efficient yet accurate detection and identification of PWOS. Accurate identification of features of the cardiac electrical signal 101, including the P wave and PWOS, can be used to more-reliably determine cardiac resynchronization therapy (CRT) parameters, providing more robust optimization and control of cardiac activation synchrony, improving patient hemodynamic response using patient-specific information. Further, in certain examples, such improvements may reduce the demand for subsequent CRT adjustment, resulting in fewer missed beats (e.g., loss of capture, etc.), improved patient outcomes, and reduced processing and power requirements. Such improvements may increase sensitivity or specificity of parameter determination, increasing data collection and storage efficiency, providing a more robust patient monitoring system, in certain examples, using less storage or data processing than existing systems. Moreover, improved detection of conditions or detection of additional conditions in sophisticated, regulatory-compliant medical systems, components, or machinery may increase the efficiency of medical system resources, improving the functioning of modern regulated technological systems and methods not capable of being performed or managed by generic computers, components, or machinery.

FIG. 2 illustrates an example system 200, such as a medical-device system including one or more AMDs, etc. The example system 200 can include a signal receiver circuit 202 and an assessment circuit 203. The signal receiver circuit 202 can be configured to receive patient information, such as physiologic information of a patient (or group of patients) from one or more sensors, such as sensor 201. The assessment circuit 203 can be configured to receive information from the signal receiver circuit 202, and to determine one or more parameters (e.g., composite physiologic parameters, stratifiers, one or more pacing parameters, etc.), such as described herein.

The assessment circuit 203 can be configured to provide an output to a user, such as to a display or one or more other user interface, the output including a score, a trend, an alert, or other indication. In other examples, the assessment circuit 203 can be configured to provide an output to another circuit, machine, or process, such as a therapy circuit 204 (e.g., a CRT therapy circuit, etc.), etc., to control, adjust, or cease a therapy of a medical device, a drug delivery system, etc., or otherwise alter one or more processes or functions of one or more other aspects of a medical-device system, such as one or more CRT parameters, etc. In an example, the therapy circuit 204 can include one or more of a stimulation control circuit and a cardiac stimulation circuit. In other examples, the therapy circuit 204 can be controlled by the assessment circuit 203, or one or more other circuits, etc.

In an example, the assessment circuit 203 can include one or more sub-circuits or processes configured to detect or measure one or more specific parameters, including, for example, one or more of: an interval between a P wave and an ensuing R wave (e.g., a PR interval); an interval between a Q wave and an ensuing T wave (e.g., a QT interval); an interval between an S wave and an ensuing T wave (e.g., an ST interval); an interval between activation of an (e.g., a right atrium (RA)) and a right ventricle (RV) (e.g., an ARV interval); an interval between an atrium (e.g., the RA) and a left ventricle (LV) (e.g., an ALV interval); a QRS width; one or more characteristics of a P wave and a heart sound characteristic (e.g., a P-S1 response, a P-S2 response, etc.); etc. In an example, a processing circuit, or one or more other components of an AMD or one or more other medical-system components, can be configured to detect or measure one or more of the parameters described herein, such as using information detected from one or more sensors, etc.

In an example, one or more aspects of the example system 200 can be a component of, or communicatively coupled to, an AMD. AMDs can be configured to monitor, detect, or treat various cardiac conditions associated with a reduced ability of a heart to sufficiently deliver blood to a body, such HF, arrhythmias, hypertension, dyssynchrony, etc. AMDs can include a single device or a plurality of medical devices or monitors implanted in a patient's body or otherwise positioned on or about the patient to monitor patient physiologic information of the patient, such as using one or more sensors (e.g., the sensor 201), the physiologic information including one or more of heart sounds, respiration (e.g., respiration rate, tidal volume, etc.), impedance (e.g., thoracic impedance, cardiac impedance, etc.), pressure (e.g., blood pressure), cardiac activity (e.g., heart rate, cardiac electrical information, etc.), physical activity, posture, plethysmography, or one or more other physiologic parameters of a patient, or to provide electrical stimulation or one or more other therapies or treatments to optimize or control contractions of the heart.

In an example, the sensor 201 can include one or more of: a respiration sensor configured to receive respiration information (e.g., a respiration rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., intrathoracic impedance sensor, transthoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); or one or more other sensors configured to receive physiologic information of the patient.

Traditional cardiac rhythm management (CRM) devices, such as pacemakers, defibrillators, or cardiac resynchronizers, include implantable or subcutaneous devices configured to be implanted in a chest of a patient, having one or more leads to position one or more electrodes or other sensors at various locations in or near the heart, such as in one or more of the atria or ventricles. Separate from, or in addition to, the one or more electrodes or other sensors of the leads, the CRM device can include one or more electrodes or other sensors (e.g., a pressure sensor, an accelerometer, a gyroscope, a microphone, etc.) powered by a power source in the CRM device. The one or more electrodes or other sensors of the leads, the CRM device, or a combination thereof, can be configured detect physiologic information from the patient, or provide one or more therapies or stimulation to the patient, such as

Implantable devices can additionally include leadless cardiac pacemakers (LCP), small (e.g., smaller than traditional implantable CRM devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable CRM device complications (e.g., required incision and pocket, complications associated with lead placement, breakage, or migration, etc.). In certain examples, an LCP can have more limited power and processing capabilities than a traditional CRM device; however, multiple LCP devices can be implanted in or about the heart to detect physiologic information from, or provide one or more therapies or stimulation to, one or more chambers of the heart. The multiple LCP devices can communicate between themselves, or one or more other implanted or external devices.

Wearable or external medical sensors or devices can be configured to detect or monitor physiologic information of the patient without required implant or an in-patient procedure for placement, battery replacement, or repair. However, such sensors and devices, in contrast to implantable medical devices, may have reduced patient compliance, increased detection noise, or reduced detection sensitivity.

AMDs may further include a pulse generator or electrical circuitry configured to electrically stimulate a heart or other excitable tissue to help restore or improve the cardiac performance or to correct or mitigate the effect of cardiac arrhythmias. Electrostimulation therapy can include CRT, such as biventricular (BiV) pacing or synchronized LV-only pacing. CRT may be indicated for patients with moderate to severe symptoms and ventricular dyssynchrony. CRT may improve cardiac function, e.g., heart pumping efficiency, increased blood flow, etc., by synchronizing contractions of the LV and RV. CRT can decrease hospitalization and morbidity associated with worsening patient conditions and improve patient quality of life (QoL).

For each AMD described above, each additional sensor can increase system cost and complexity, reduce system reliability, or increase the power consumption and reduce the usable life of the ambulatory device. Accordingly, it can be beneficial to use a single sensor to determine multiple types of physiologic information, or a smaller number of sensors to measure a larger number of different types of physiologic information. For example, it can be beneficial to detect atrial cardiac electrical information without a lead or an electrode in, or in contact with, the atria.

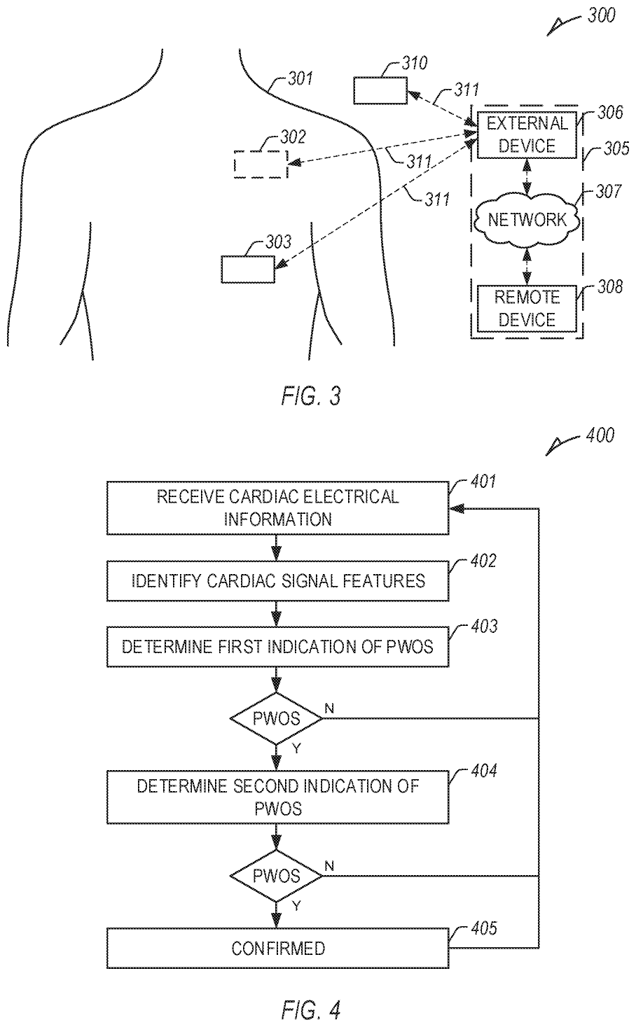

FIG. 3 illustrates an example patient management system 300 and portions of an environment in which the system 300 may operate. The patient management system 300 can perform a range of activities, including remote patient monitoring and diagnosis of a disease condition. Such activities can be performed proximal to a patient 301, such as in a patient home or office, through a centralized server, such as in a hospital, clinic, or physician office, or through a remote workstation, such as a secure wireless mobile computing device.

The patient management system 300 can include one or more ambulatory devices, an external system 305, and a communication link 311 providing for communication between the one or more ambulatory devices and the external system 305. The one or more ambulatory devices can include an implantable medical device (IMD) 302, a wearable medical device 303, or one or more other implantable, leadless, subcutaneous, external, wearable, or ambulatory medical devices configured to monitor, sense, or detect information from, determine physiologic information about, or provide one or more therapies to treat various cardiac conditions of the patient 301, such as AF, congestive heart failure (CHF), hypertension, or one or more other cardiac or non-cardiac conditions (e.g., dehydration, hemorrhage, high blood pressure, renal dysfunction, etc.).

In an example, the IMD 302 can include one or more traditional cardiac rhythm management (CRM) devices, such as a pacemaker or defibrillator, implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 301. In another example, the IMD 302 can include a monitor implanted, for example, subcutaneously in the chest of patient 301.

The IMD 302 can include an assessment circuit configured to detect or determine specific physiologic information of the patient 301, or to determine one or more conditions or provide information or an alert to a user, such as the patient 301 (e.g., a patient), a clinician, or one or more other caregivers. The IMD 302 can alternatively or additionally be configured as a therapeutic device configured to treat one or more medical conditions of the patient 301. The therapy can be delivered to the patient 301 via the lead system and associated electrodes or using one or more other delivery mechanisms. The therapy can include anti-arrhythmic therapy to treat an arrhythmia or to treat or control one or more complications from arrhythmias, such as syncope, CHF, or stroke, among others. In other examples, the therapy can include delivery of one or more drugs to the patient 301 using the IMD 302 or one or more of the other ambulatory devices. Examples of the anti-arrhythmic therapy include pacing, cardioversion, defibrillation, neuromodulation, drug therapies, or biological therapies, among other types of therapies. In other examples, therapies can include CRT for rectifying dyssynchrony and improving cardiac function in CHF patients. In some examples, the MID 302 can include a drug delivery system, such as a drug infusion pump to deliver drugs to the patient for managing arrhythmias or complications from arrhythmias, hypertension, or one or more other physiologic conditions. In yet other examples, the MID 302 can include a therapy circuit or module configured to treat hypertension (e.g., a neuro-stimulation therapy circuit, a drug delivery therapy circuit, a stimulation therapy circuit, etc.).

The wearable medical device 303 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.). The wearable medical device 303 can include an optical sensor configured to detect a photoplethysmogram (PPG) signal on a wrist, finger, or other location on the patient 301. In other examples, the wearable medical device 303 can include an acoustic sensor or accelerometer to detect acoustic information (e.g., heart sounds) or the sound or vibration of blood flow, an impedance sensor to detect impedance variations associated with changes in blood flow or volume, a temperature sensor to detect temperature variation associated with blood flow, a laser Doppler vibrometer or other pressure, strain, or physical sensor to detect physical variations associated with blood flow, etc.

The patient management system 300 can include, among other things, a respiration sensor configured to receive respiration information (e.g., a respiration rate, a respiration volume (tidal volume), etc.), a heart sound sensor configured to receive heart sound information, a thoracic impedance sensor configured to receive impedance information, a cardiac sensor configured to receive cardiac electrical information, an activity sensor configured to receive information about a physical motion (e.g., activity, posture, etc.), a plethysmography sensor, or one or more other sensors configured to receive physiologic information of the patient 301.

The external system 305 can include a dedicated hardware/software system, such as a programmer, a remote server-based patient management system, or alternatively a system defined predominantly by software running on a standard personal computer. The external system 305 can manage the patient 301 through the IMD 302 or one or more other ambulatory devices connected to the external system 305 via a communication link 311. In other examples, the IMD 302 can be connected to the wearable device 303, or the wearable device 303 can be connected to the external system 305, via the communication link 311. This can include, for example, programming the IMD 302 to perform one or more of acquiring physiological data, performing at least one self-diagnostic test (such as for a device operational status), analyzing the physiological data to detect a cardiac arrhythmia, or optionally delivering or adjusting a therapy to the patient 301. Additionally, the external system 305 can send information to, or receive information from, the IMD 302 or the wearable device 303 via the communication link 311. Examples of the information can include real-time or stored physiological data from the patient 301, diagnostic data, such as detection of cardiac arrhythmias or events of worsening heart failure, responses to therapies delivered to the patient 301, or device operational status of the MID 302 or the wearable device 303 (e.g., battery status, lead impedance, etc.). The communication link 311 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

By way of example and not limitation, the external system 305 can include an external device 306 in proximity of the one or more ambulatory devices, and a remote device 308 in a location relatively distant from the one or more ambulatory devices, in communication with the external device 306 via a communication network 307. Examples of the external device 306 can include a medical device programmer.

The remote device 308 can be configured to evaluate collected patient or patient information and provide alert notifications, among other possible functions. In an example, the remote device 308 can include a centralized server acting as a central hub for collected data storage and analysis. The server can be configured as a uni-, multi-, or distributed computing and processing system. The remote device 308 can receive data from multiple patients. The data can be collected by the one or more ambulatory devices, among other data acquisition sensors or devices associated with the patient 301. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory devices, such as the IMD. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiological data associated with the detected medical event to physiological data associated with the historical alerts.

The remote device 308 may additionally include one or more locally configured clients or remote clients securely connected over the communication network 307 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 308, including the server and the interconnected clients, may also execute a follow-up scheme by sending follow-up requests to the one or more ambulatory devices, or by sending a message or other communication to the patient 301 (e.g., the patient), clinician or authorized third party as a compliance notification.

The communication network 307 can provide wired or wireless interconnectivity. In an example, the communication network 307 can be based on the Transmission Control Protocol/Internet Protocol (TCP/IP) network communication specification, although other types or combinations of networking implementations are possible. Similarly, other network topologies and arrangements are possible.

One or more of the external device 306 or the remote device 308 can output the detected medical events to a system user, such as the patient or a clinician, or to a process including, for example, an instance of a computer program executable in a microprocessor. In an example, the process can include an automated generation of recommendations for anti-arrhythmic therapy, or a recommendation for further diagnostic test or treatment. In an example, the external device 306 or the remote device 308 can include a respective display unit for displaying the physiological or functional signals, or alerts, alarms, emergency calls, or other forms of warnings to signal the detection of arrhythmias. In some examples, the external system 305 can include an external data processor configured to analyze the physiological or functional signals received by the one or more ambulatory devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory devices or the external system 305 can be implemented using hardware, software, firmware, or combinations thereof. Portions of the one or more ambulatory devices or the external system 305 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The patient management system 300 can include a therapy device (e.g., a therapy circuit 309, etc.), such as a drug delivery device configured to provide therapy or therapy information (e.g., dosage information, etc.) to the patient 301, such as using information from one or more of the ambulatory devices. In other examples, one or more of the ambulatory devices can be configured to provide therapy or therapy information to the patient 301. The therapy device can be configured to send information to or receive information from one or more of the ambulatory devices or the external system 305 using the communication link 311. In an example, the one or more ambulatory devices, the external device 306, or the remote device 308 can be configured to control one or more parameters of the therapy device 310.

The external system 305 can allow for programming the one or more ambulatory devices and can receives information about one or more signals acquired by the one or more ambulatory devices, such as can be received via a communication link 311. The external system 305 can include a local external IMD programmer. The external system 305 can include a remote patient management system that can monitor patient status or adjust one or more therapies such as from a remote location.

The patient chronic condition-based HF assessment circuit, or other assessment circuit, may be implemented at the external system 305, which can be configured to perform HF risk stratification such as using data extracted from the one or more ambulatory devices or data stored in a memory within the external system 305. Portions of patient chronic condition-based HF or other assessment circuit may be distributed between the one or more ambulatory devices and the external system 305.

FIG. 4 illustrates a method 400 of detecting P wave oversensing (PWOS), such as using one or more AMDs, a medical device system, etc. At 401, cardiac electrical information can be received, such as using a signal receiver circuit. In an example, cardiac electrical information, such as a cardiac electrical signal, can be sensed or detected using one or more sensors, such as one or more electrodes, etc. In an example, the signal receiver circuit can receive the cardiac electrical signal, one or more features from the cardiac signal feature, or other cardiac signal information, such as from one or more sensors, AMDs, etc. The received cardiac electrical information can be stored, such as temporarily in a buffer (e.g., a circular buffer, etc.), or more permanently in non-volatile or volatile memory of an AMD or one or more medical system components, etc.

At 402, cardiac signal features can be identified in a first portion of the received cardiac electrical information, such as using the assessment circuit. In certain examples, cardiac signal features can include a timing, amplitude, or one or more other physical characteristics of a P wave, an R wave, a PR interval, or one or more other cardiac signal features, and the first portion of the received cardiac electrical information can include one or more cardiac cycles.

At 403, a first indication of PWOS can be determined using a pattern of identified cardiac signal features, such as using the assessment circuit. In an example, the pattern can include one or more different patterns, such as timing or amplitude variation of one or more cardiac signal features. For example, a substantially long or short durations between successive R wave and/or P wave detections (e.g., RR intervals. PR intervals, PP intervals, etc.) can be indicative of detection error or noise. In certain examples, substantially long or short durations can represent a 50% rate or interval variation or greater, such as with respect to a previous or subsequent duration or short-term average including the duration in question, etc. Successive detections in a short time period (e.g., satisfying the above rate or interval variation, etc.) can be indicative of PWOS. A long-short pattern, such as a P wave detection followed quickly by an R wave detection, or an R wave detected in refractory or a noise period, can be indicative of MOS.

In an example, the first indication can include a persistent or sustained indication of PWOS over multiple cardiac cycles (e.g., consecutive cycles, a certain number of consecutive cycles, etc.), or above a threshold number of indications of PWOS over a specified interval or number of cardiac cycles (e.g., X of Y cycles). The determined first indication of PWOS can trigger determination of a second indication of PWOS, in certain examples, on the same or at least a portion of the same information that triggered the first indication.

In an example, a normal range of PR interval can include 120-200 ms. In other examples, the normal range of PR interval can be: a population-based. PR interval, such as for a specified disease state or physiologic condition associated with the patient, etc.; a rate-dependent PR interval, such as a PR interval that varies according to the heart rate for that interval or one or more intervals around the specific PR interval; a patient-specific PR interval; or a combination thereof. Timing variations can include determination of normal and abnormal PR intervals. An abnormal PR interval can include a PR interval outside the normal range. In other examples, an abnormal PR interval can include a difference between successive PR intervals above a threshold (e.g., greater than 50% of the prior interval, etc.), or a combination of the difference between successive PR intervals above a threshold and the PR interval outside the normal range.

In other examples, amplitude variations can include determinations of small or tall amplitudes of cardiac signal features, such as determined P waves, R waves, etc. Small and tall determinations can be made using comparisons to population-based amplitudes, patient-specific long or short-term amplitudes, posture-based population or patient-specific amplitudes. A normal range can be determined from one or more of the population-based, patient-specific, or posture-based amplitudes. An abnormal amplitude can include an amplitude for a specified, determined cardiac signal feature outside of the normal range for that specified, determined cardiac signal feature. In other examples, an abnormal amplitude can include a difference between successive amplitudes of a specified, determined cardiac signal feature above a threshold (e.g., greater than 50% of the prior amplitude, etc.), or a combination of the difference between successive amplitudes above a threshold and the amplitudes outside the normal range.

If, at 403, a first indication of PWOS is not determined, process can return to 401. If, at 403, a first indication of PWOS is determined, a second indication of PWOS can be determined at 404, different than the first indication of PWOS. In an example, the determination of the second indication of PWOS can be more resource-intensive than determination of the first indication of PWOS, and can have a higher sensitivity, specificity, or sensitivity and specificity than determination of the first indication. Determination of the second indication of PWOS can confirm the first indication of PWOS. For example, in response to a determined first indication of PWOS, a morphology analysis of the determined first indication of PWOS can be performed (e.g., using a morphology check against one or more templates, such as an R wave template, a normal sinus rhythms (NSR) template, a beat-to-beat template, etc.) to confirm the determined first indication of PWOS. The one or more templates can include patient-specific or population-based templates of cardiac signal features, such as confirmed by a process or user.

In an example, a patient-specific or population-based template can be used to confirm or reject potential cardiac signal features, such as using a morphology analysis (e.g., of a shape, one or more frequency components, etc.) of the potential identified cardiac signal feature to the patient-specific or population-based template. For example, morphology analysis can include determination of similarity between a potential cardiac signal feature and a template. If the similarity of one or more aspect or criteria of the potential cardiac signal feature is determined above a threshold, the potential cardiac signal feature can be confirmed. If it is determined below a threshold, the potential cardiac signal feature can fail the morphology check. For example, the morphology of an identified potential P wave can be checked against one or more of a P wave template or a template for one or more other cardiac signal features, such as an R wave, etc. The morphology of an identified potential R wave can be checked against one or more of an R wave template or a template for one or more other cardiac signal features, such as a P wave, etc. If an identified cardiac signal feature fails a morphology check against a template for the respective identified cardiac signal feature, it can be checked against a template for one or more other cardiac signal features. If a mis-identified cardiac signal feature is determined, such as using such morphology check, it can be corrected and used to determine one or more other features. For example, if a P wave is mis-identified as an R wave, and a morphology check correctly identifies the mis-identified cardiac signal feature as a P wave, the PR interval, RR interval, or one or more other feature including the now-properly-identified cardiac signal features, can be corrected. If such features are being used to modify or adjust one or more pacing or therapy parameters, adjustments can be made or determined, or a notification to a user can be provided.

If, at 404, a second indication of PWOS is not determined, process can return to 401. If, at 404, a second indication of PWOS is determined, then PWOS can be confirmed at 405, and the system or one or more components thereof can respond accordingly. In an example, if a determined initial PWOS event is confirmed using the morphology determination, an alert or notification can be provided to a user or a process to change programming of one or more medical devices, such as to change a sensing vector of an AMD or sensor configured to detect the cardiac electrical information (e.g., the cardiac electrical signal, etc.) to change a sensitivity of detection, etc. In other examples, confirmation of a PWOS can trigger storage of at least a portion of the cardiac electrical information, such as cardiac electrical information including or preceding the confirmed PWOS for further processing. In other examples, the number of confirmed PWOS events, or initial PWOS events, can be counted, and an alert can be issued using the confirmed PWOS events, the initial PWOS events, or a combination thereof, such as to provide an alert or notification to a user or a process, etc. For example, if a number of initial PWOS events are detected and not confirmed, an alert can be issued to alter one or more sensing or detection parameters. If a number of confirmed PWOS events are detected (e.g., ten consecutive PWOS events, etc.), an alert can be issued to alter one or more sensing or detection parameters.

In certain examples, one or both of the first or second determined indications of PWOS can trigger more permanent storage of the cardiac electrical information, or a portion of the cardiac electrical information associated with one or both of the first or second determined indications. In an example, if the second determined indication confirms the first determined indication, the cardiac electrical information can be stored or marked for storage, such as in non-volatile memory. If the second determined indication does not confirm the first determined indication, some or all of the cardiac electrical information associated with the first determined indication can be discarded or marked for deletion. Such storage can reduce storage requirements of the system, etc.

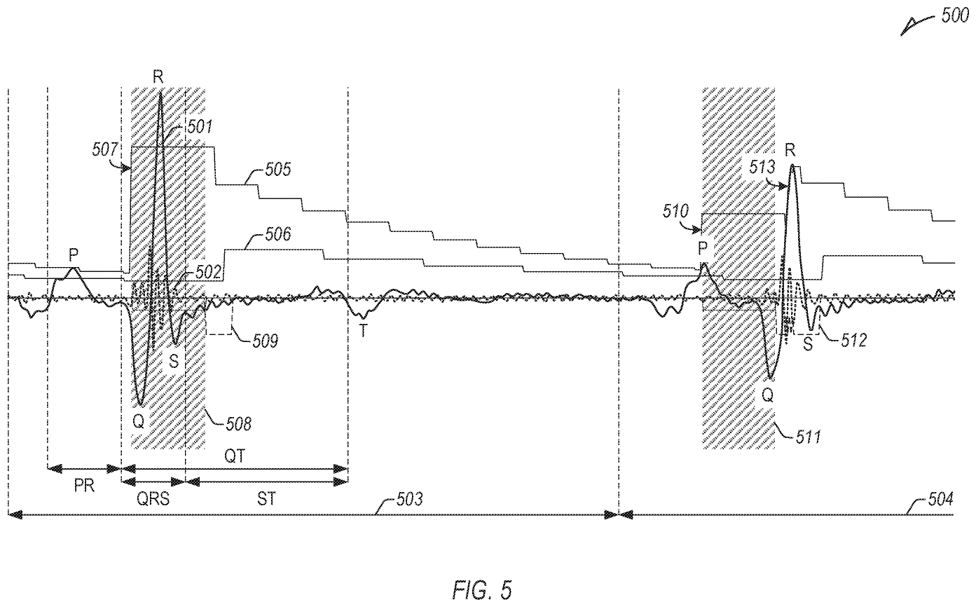

FIG. 5 illustrates an example relationship 500 between features of a cardiac signal 501 (e.g., an electrogram (EGM), etc.) and a high-pass-filtered cardiac signal 502 of a patient over multiple physiologic cycles of the patient including first and second cardiac cycles 503, 504. The relationships 500 include different cardiac electrical features or parameters, including the P wave (P), the QRS complex (including the Q wave (Q), the R wave (R), and the S wave (S)), the T wave (I), etc., and different timing intervals between different features of the cardiac electrical signal 501, including a PR interval (PR), a QRS interval (QRS), a QT interval (QT), an ST interval (ST), etc.

The relationship 500 includes two detection thresholds: a first detection threshold 505 based on the cardiac signal 501 and a second detection threshold 506 based on the high-pass-filtered cardiac signal 502. A detection occurs when the cardiac signal 501 exceeds the greater of the first and second detection thresholds 505, 506. In the first cardiac cycle 503, a first detection occurs at 507 when the absolute value of the Q wave exceeds the first detection threshold 505, triggering a first refractory period 508. No detections can occur in a refractory period. A first re-triggerable noise period 509 follows the first refractory period 508, such as to avoid mis-detection of the T wave by tailing features of the QRS complex. One or more other detections can separate or detect the individual features of the QRS complex, such as distinguishing the R wave from the Q wave, etc.

In the second cardiac cycle 504, a second detection occurs at 510 when the P wave exceeds the first detection threshold 505, triggering a second refractory period 511. This second detection at 510 is a mis-detected QRS complex. A second re-triggerable noise period 512 follows the second refractory period 511. After the second refractory period 511, the first detection threshold 505 drops, but a third detection occurs at 513 when the R wave exceeds the first detection threshold 505. The first detection threshold 505 increases each time it is exceeded, re-triggering the re-triggerable noise period 512. Accordingly, two QRS complexes are detected in the second cardiac cycle 504. If such mis-detections are not determined, they can appear to a sensor, an AMD, or one or more other medical system components as two fast beats. Further, an otherwise detectable and accurate PR interval, and RR interval, may be missed because of such mis-detection.

Portions of the cardiac signal 501 can be stored in memory in one or more AMD or medical system components (e.g., in a memory circuit, etc.). In certain examples, a circular buffer can store an amount of preceding cardiac electrical information, in certain examples including the cardiac signal 501. Certain events can trigger more permanent storage outside of the circular buffer, such as in volatile or non-volatile memory. In an example, an abnormal pattern of cardiac signal features, such as one or more rates or intervals outside a threshold amount (e.g., a threshold above or below patient-specific short or long-term averages, pre-define or pre-set intervals, etc.) can trigger further processing or storage of the cardiac electrical information including the abnormal pattern. In certain examples, a number of intervals outside the threshold amount, such as successive intervals or a threshold number in a previous number of intervals, are required to trigger further processing or storage of the cardiac electrical information.

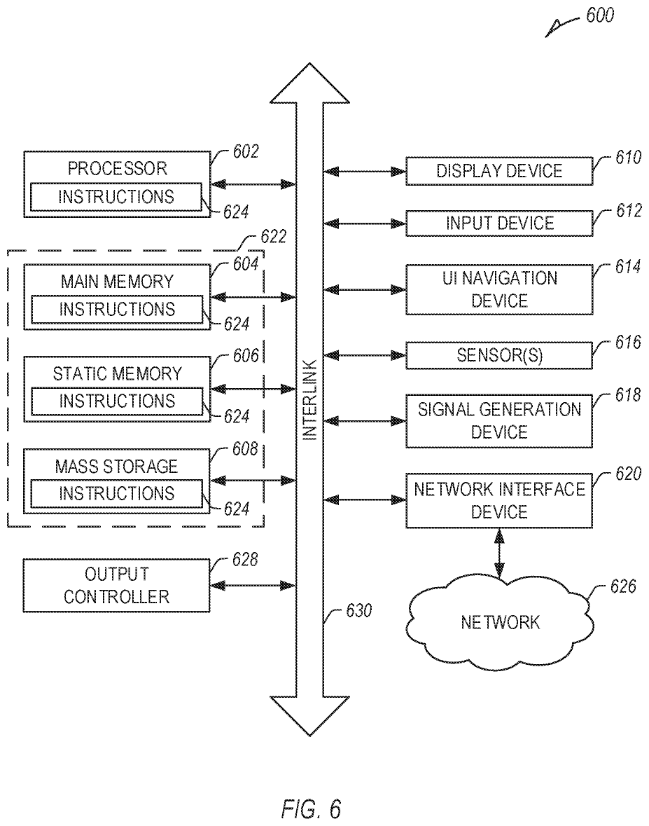

FIG. 6 illustrates a block diagram of an example machine 600 upon which any one or more of the techniques (e.g., methodologies) discussed herein may perform. Portions of this description may apply to the computing framework of one or more of the medical devices described herein, such as the IMD, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 600. Circuitry (e.g., processing circuitry) is a collection of circuits implemented in tangible entities of the machine 600 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 600 follow.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 600 may include a hardware processor 602 (e.g., a central processing unit (CPU), a graphics processing unit (GPU), a hardware processor core, or any combination thereof), a main memory 604, a static memory (e.g., memory or storage for firmware, microcode, a basic-input-output (BIOS), unified extensible firmware interface (UM), etc.) 606, and mass storage 608 (e.g., hard drive, tape drive, flash storage, or other block devices) some or all of which may communicate with each other via an interlink (e.g., bus) 630. The machine 600 may further include a display unit 610, an alphanumeric input device 612 (e.g., a keyboard), and a user interface (UT) navigation device 614 (e.g., a mouse). In an example, the display unit 610, input device 612, and UT navigation device 614 may be a touch screen display. The machine 600 may additionally include a signal generation device 618 (e.g., a speaker), a network interface device 620, and one or more sensors 616, such as a global positioning system (GPS) sensor, compass, accelerometer, or one or more other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.

Registers of the processor 602, the main memory 604, the static memory 606, or the mass storage 608 may be, or include, a machine-readable medium 622 on which is stored one or more sets of data structures or instructions 624 (e.g., software) embodying or utilized by any one or more of the techniques or functions described herein. The instructions 624 may also reside, completely or at least partially, within any of registers of the processor 602, the main memory 604, the static memory 606, or the mass storage 608 during execution thereof by the machine 600. In an example, one or any combination of the hardware processor 602, the main memory 604, the static memory 606, or the mass storage 608 may constitute the machine-readable medium 622. While the machine-readable medium 622 is illustrated as a single medium, the term “machine-readable medium” may include a single medium or multiple media (e.g., a centralized or distributed database, and/or associated caches and servers) configured to store the one or more instructions 624.

The term “machine-readable medium” may include any medium that is capable of storing, encoding, or carrying instructions for execution by the machine 600 and that cause the machine 600 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may be further transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 600, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A system; comprising:
a signal receiver circuit configured to receive cardiac electrical information of a patient;
an assessment circuit configured to:
identify cardiac signal features in a first portion of the received cardiac electrical information;
determine a first indication of P wave oversensing (MOS) in the first portion of the received cardiac electrical information using a pattern of identified cardiac signal features; and
in response to the determined first indication of PWOS in the first portion of the received cardiac electrical information, determine a second indication of PWOS in the first portion of the received cardiac electrical information using a morphology of the first portion of the cardiac electrical information.

2. The system of claim 1, wherein the first portion of the received cardiac electrical information includes a first cardiac cycle of the patient, and
wherein the cardiac signal features include at least one of a P wave or an R wave of the first cardiac cycle.

3. The system of claim 1, wherein the first portion of the received cardiac electrical information includes multiple cardiac cycles of the patient, and
wherein the cardiac signal features include at least one of a P wave of the multiple cardiac cycles or an R wave of the multiple cardiac cycles.

4. The system of claim 1, wherein the pattern of identified cardiac signal features includes a pattern of amplitudes of the identified cardiac signal features or timings of or between the identified cardiac signal features.

5. The system of claim 4, wherein the pattern of identified cardiac signal features includes a pattern of timings between identified cardiac signal features.

6. The system of claim 1, wherein, to determine the second indication of PWOS, the assessment circuit is configured to compare a morphology of at least one identified cardiac signal feature associated with the determined indication of PWOS to a template for the identified cardiac signal feature to confirm the determined first indication of PWOS.

7. The system of claim 6, wherein the template includes at least one of an R wave template or a P wave template.

8. The system of claim 1, wherein the system is a medical-device system, comprising:
a cardiac stimulation circuit configured to provide a cardiac stimulation signal to stimulate a heart of the patient; and
a stimulation control circuit configured to adjust the cardiac stimulation signal using the determined second indication of PWOS in the first portion of the received cardiac electrical information.

9. The system of claim 8, comprising:
multiple electrodes configured to provide stimulation to the heart of the patient using the cardiac stimulation signal from the cardiac stimulation circuit and to detect the cardiac electrical information of the patient.

10. A method, comprising:
receiving, using a signal receiver circuit, cardiac electrical information of a patient;
identifying, using an assessment circuit, cardiac signal features in a first portion of the received cardiac electrical information;
determining; using the assessment circuit:
a first indication of P wave oversensing (MVOS) in the first portion of the received cardiac electrical information using a pattern of identified cardiac signal features; and
in response to the determined first indication of MOS in the first portion of the received cardiac electrical information, a second indication of PWOS in the first portion of the received cardiac electrical information using a morphology of the first portion of the cardiac electrical information.

11. The method of claim 10, wherein the first portion of the received cardiac electrical information includes a first cardiac cycle of the patient, and
wherein the cardiac signal features includes at least one of a P wave or an R wave of the first cardiac cycle.

12. The method of claim 10, wherein the first portion of the received cardiac electrical information includes multiple cardiac cycles of the patient, and
wherein the cardiac signal features include at least one of a P wave of the multiple cardiac cycles or an R wave of the multiple cardiac cycles.

13. The method of claim 10, wherein the pattern of identified cardiac signal features includes a pattern of amplitudes of the identified cardiac signal features or timings of or between the identified cardiac signal features.

14. The method of claim 13, wherein the pattern of identified cardiac signal features includes a pattern of timings between identified cardiac signal features.

15. The method of claim 10, wherein determining the second indication of PWOS includes comparing a morphology of at least one identified cardiac signal feature associated with the determined indication of PWOS to a template for the identified cardiac signal feature to confirm the determined first indication of PWOS.

16. The method of claim 15, wherein the template includes at least one of an R wave template or a P wave template.

17. The method of claim 10, comprising:
providing, using a cardiac stimulation circuit, a cardiac stimulation signal to stimulate a heart of the patient; and
adjusting, using a stimulation control circuit, the cardiac stimulation signal using the determined second indication of PWOS in the first portion of the received cardiac electrical information.

18. The method of claim 17, comprising:
providing, using multiple electrodes, stimulation to the heart of the patient using the cardiac stimulation signal from the cardiac stimulation circuit; and
detecting the cardiac electrical information of the patient using the multiple electrodes.

19. A system, comprising:
a signal receiver circuit configured to receive cardiac electrical information of a patient;
an assessment circuit configured to:
identify cardiac signal features in a first portion of the received cardiac electrical information, wherein the first portion includes a first cardiac cycle of the patient and the cardiac signal features include at least one of a P wave or an R wave of the first cardiac cycle;
determine a first indication of P wave oversensing (PWOS) in the first portion of the received cardiac electrical information using a pattern of identified cardiac signal features; and
in response to the determined first indication of PWOS in the first portion of the received cardiac electrical information, determine a second indication of PWOS in the first portion of the received cardiac electrical information using a morphology of the first portion of the cardiac electrical information.

20. The system of claim 19, wherein the pattern of identified cardiac signal features includes a pattern of amplitudes of the identified cardiac signal features or timings of or between the identified cardiac signal features,
wherein, to determine the second indication of PWOS, the assessment circuit is configured to compare a morphology of at least one identified cardiac signal feature associated with the determined indication of MVOS to a template for the identified cardiac signal feature to confirm the determined first indication of PWOS, and
wherein the template includes at least one of an R wave template or a P wave template.
